(19) United States
(12) Patent Application Publication
(10) Pub. No.: US 2005/0137252 A1
(43) Pub. Date: Jun. 23, 2005

(54) Production of dihydronepetalactone

(76) Inventor: Mark A. Scialdone, Oxford, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/017,254

(22) Filed: Dec. 20, 2004

Related U.S. Application Data
(60) Provisional application No. 60/531,775, filed on Dec. 22, 2003.

(51) Int. Cl. A61K 31/366; C07D 311/02

(52) U.S. Cl. 514/457; 549/285

(57) ABSTRACT

Disclosed is a process for preparing dihydronepetalactone by way of a nepetalic acid intermediate to yield a stereospecific product, and compositions and articles made from the product.

This application claims the benefit of U.S. Provisional Application No. 60/531,775, filed Dec. 22, 2003, which is incorporated in its entirety as a part hereof for all purposes.

### FIELD OF THE INVENTION

This invention relates to a process for preparing dihydronepetalactone by way of a nepetalic acid intermediate to yield a stereospecific product.

### BACKGROUND OF THE INVENTION

Many plant species belonging to the family Labiatae (Lamiaceae) produce essential oils (aromatic oils), some of which may be used as natural sources of insect repellent and fragrant chemicals [Hay, R. K. M. and Svoboda, K. P., Botany, In “Volatile Oil Crops: their biology, chemistry and production”; Hay, R. K. M. and Waterman, P. G. (eds.); Longman Group UK Limited (1993)]. Plants of the genus Nepeta (catmints) are included as members of this family, and produce an essential oil that is a minor item of commerce primarily in the form of catnip in cat toys. This oil is very rich in a class of monoterpenoid compounds known as iridoids [Inouye, H., Iridoids, Methods in Plant Biochemistry 7:99-143 (1991)], more specifically the methylcyclopentanoid nepetalactones [Clark, L. J. et al., The Plant Journal, 11:1387-1393 (1997)] and derivatives.

Four stereoisomers of nepetalactone are known, and they are represented by the structures set forth in FIG. 1. Three of the four are known to exist in nature. The cis, trans isomer, FIG. 1(a), is the predominant component of the essential oil of nepeta cateria, present to about 80%. Other species of the genus nepeta are believed to have predominantly the trans, cis isomer.

Nepetalactone may be converted to dihydronepetalactones (DHN), and processes for producing DHN by catalytic hydrogenation of nepetalactone are described in Regnier, R. E. et al., Phytochemistry 6:1281-1289 (1967). Manzer, in WO 03/084946, discloses further catalytic routes to DHN from nepetalactone. The eight possible stereoisomers of DHN are shown in FIG. 2.

DHN is known to exhibit insect repellent characteristics. See, for example, Jefson, M., et al., J. Chemical Ecology 9:159-180 (1983). Jefson, op.cit., isolates DHN from the secretions of certain species of beetles, and identifies one specific stereoisomer obtained as (1R,5R,6R,9S)-5,9-dimethyl-3-oxabicyclo[4.3.0]nonan-2-one (Structure F in FIG. 2). Jefson also employs the techniques of Wolinsky et al to synthesize by a laboratory route the same diastereomer.

Hallahan, in WO 03/079786, discloses that DHN exerts a repellent effect on the common insect pests of human society. Also disclosed by Hallahan is that different stereoisomers of DHN, and mixtures thereof, exhibit different degrees of insect repellency to different species of insects. Achieving an optimum degree of insect repellency for any particular purpose thus necessitates screening various stereoisomers in isolation and in mixtures of varying proportions.

Only certain stereoisomers of DHN are available by conventional means, however. For example, referring to FIG. 2, it has been found that the catalytic hydrogenation of trans, cis nepetalactone produces a high yield of DHN Structure B, (1S,5R,6R,9S)-5,9-dimethyl-3-oxabicyclo[4,3,0]nonan-2-one. On the other hand, it has also been found that catalytic hydrogenation of cis, trans nepetalactone, the most prevalent and easily purified isomer, results in an approximately 7:1 mixture of Structure E {(1R,5S,6R,9S)-5,9-dimethyl-3-oxabicyclo[4,3,0]nonan-2-one} and Structure F {(1R,5R,6R,9S)-5,9-dimethyl-3-oxabicyclo[4,3,0]nonan-2-one}, respectively. This diastereomeric mixture is not readily separable.

It may thus be seen that the synthetic routes taught in the art for preparing DHN from nepetalactones are based upon catalytic hydrogenation of mixtures of nepetalactones containing predominantly the cis, trans stereoisomer. To a lesser degree the art also teaches the hydrogenation of the trans, cis stereoisomer, again from a mixture of nepetalactones containing in this case predominantly the trans, cis nepetalactone. Hydrogenation of the cis, trans nepetalactone by the processes of the art produces a 7:1 diastereomeric mixture of the isomers shown as Structures E and F, respectively, in FIG. 2. This mixture is not susceptible to separation by ordinary means. Hydrogenation of the trans, cis nepetalactone produces a single diastereomer, shown as Structure B in FIG. 2.

The catalytic routes to the isomers described above provide economy and efficiency of production with a high degree of selectivity to those particular DHN isomers. It would be desirable, however, to be able to easily produce a wider variety of DHN isomers because the application of DHN to its full range of uses will require that more than a few isomers be readily available in commercial quantities. The known catalytic methods, in addition to having a focus restricted to just certain isomers, also possess the typical, undesirable aspects of catalysis, such as possible contamination of the final product, and the need to recover and recycle the catalyst. A need thus remains for a process that is not dependent on catalysis to easily and efficiently produce a variety of isomers of DHN.

The method of the present invention provides a novel synthetic route from nepetalactone to DHN diastereomers, and mixtures thereof, not heretofore available from naturally occurring nepetalactones, thereby greatly expanding the number of practical formulations that are useful in the many applications of DHN such as fragrances and insect repellents.

### SUMMARY OF THE INVENTION

One embodiment of this invention is a process for preparing a dihydronepetalactone, represented schematically as Structure II in the reaction scheme, by
(a) contacting nepetalactone, represented schematically as Structure I, with an aqueous base;
(b) contacting the product of step (a) with an acid to form nepetalic acid, represented schematically as Structure III;
(c) contacting the nepetalic acid with a reducing agent to form dihydronepetalactone.

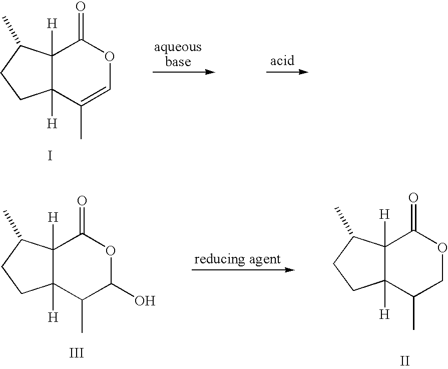

The nepetalactone may, for example, be cis, trans nepetalactone ((3S,4R,4aR,7S,7aR)-3-hydroxy-4,7-dimethylhexahydrocyclopenta[c]pyran-1(3H)-one), represented by Structure I(a), 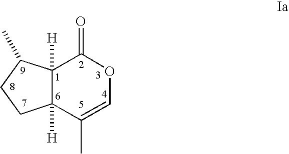
and a dihydronepetalactone so produced may be (9S,1R,5R,6R)-5,9-dimethyl-3-oxabicyclo[4.3.0]nonan-2-one, represented by Structure F

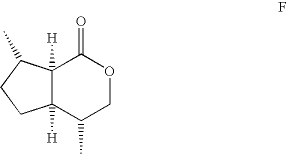

Another embodiment of this invention is a composition of matter that includes (a-1) the single diastereomer of dihydronepetalactone (9S,1R,5R,6R)-5,9-dimethyl-3-oxabicyclo[4.3.0]nonan-2-one, or (a-2) a mixture of diastereomers of dihydronepetalactone whereof at least 50% thereof is (9S,1R,5R,6R)-5,9-dimethyl-3-oxabicyclo[4.3.0]nonan-2-one; and (b) a carrier. This composition is useful in insect repellant and fragrance applications.

A further embodiment of this invention is an article of manufacture that incorporates the single diastereomer of dihydronepetalactone (9S,1R,5R,6R)-5,9-dimethyl-3-oxabicyclo[4.3.0]nonan-2-one, or a mixture of diastereomers of dihydronepetalactone whereof at least 50% thereof is (9S,1R,5R,6R)-5,9-dimethyl-3-oxabicyclo[4.3.0]nonan-2-one.

Yet another embodiment of this invention is a method of repelling one or more insects from a human, animal or inanimate host by exposing the insect(s) to the single diastereomer of dihydronepetalactone (9S,1R,5R,6R)-5,9-dimethyl-3-oxabicyclo[4.3.0]nonan-2-one, or to a mixture of diastereomers of dihydronepetalactone whereof at least 50% thereof is (9S,1R,5R,6R)-5,9-dimethyl-3-oxabicyclo[4.3.0]nonan-2-one, or to a composition thereof.

Yet another embodiment of this invention is the use of the single diastereomer of dihydronepetalactone (9S,1R,5R,6R)-5,9-dimethyl-3-oxabicyclo[4.3.0]nonan-2-one, or a mixture of diastereomers of dihydronepetalactone whereof at least 50% thereof is (9S,1R,5R,6R)-5,9-dimethyl-3-oxabicyclo[4.3.0]nonan-2-one, to repel insects from a human, animal or inanimate host.

Yet another embodiment of this invention is the use of the single diastereomer of dihydronepetalactone (9S,1R,5R,6R)-5,9-dimethyl-3-oxabicyclo[4.3.0]nonan-2-one, or a mixture of diastereomers of dihydronepetalactone whereof at least 50% thereof is (9S,1R,5R,6R)-5,9-dimethyl-3-oxabicyclo[4.3.0]nonan-2-one, as a fragrance compound or as a topical treatment for skin.

Yet another embodiment of this invention is a method of fabricating an insect repellent composition, or an insect repellent article of manufacture, by forming the composition from, or incorporating into the article, the single diastereomer of dihydronepetalactone (9S,1R,5R,6R)-5,9-dimethyl-3-oxabicyclo[4.3.0]nonan-2-one, or a mixture of diastereomers of dihydronepetalactone whereof at least 50% thereof is (9S,1R,5R,6R)-5,9-dimethyl-3-oxabicyclo[4.3.0]nonan-2-one.

Yet another embodiment of this invention is a method of fabricating a composition to be applied to skin, or a fragrant article of manufacture, by forming the composition from, or incorporating into the article, the single diastereomer of dihydronepetalactone (9S,1R,5R,6R)-0,5,9-dimethyl-3-oxabicyclo[4.3.0]nonan-2-one, or a mixture of diastereomers of dihydronepetalactone whereof at least 50% thereof is (9S,1R,5R,6R)-5,9-dimethyl-3-oxabicyclo[4.3.0]nonan-2-one. The composition to be applied to skin may have fragrant or other therapeutic properties.

### BRIEF DESCRIPTION OF THE DRAWINGS

FIG. 1 shows the chemical structures of the naturally-occurring iridoid (methylcyclopentanoid) nepetalactones.

FIG. 2 shows the eight possible diastereomers of dihydronepetalactones (DHN).

FIG. 3 shows the results of Example 3.

### DETAILED DESCRIPTION OF THE INVENTION

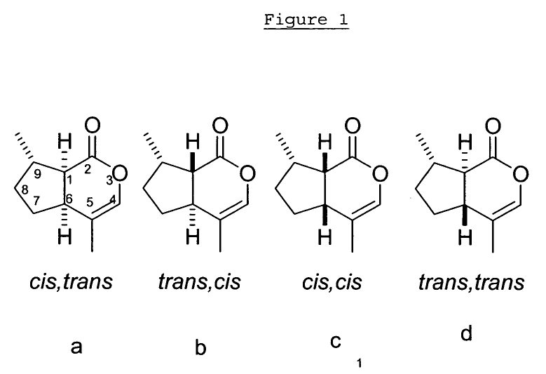

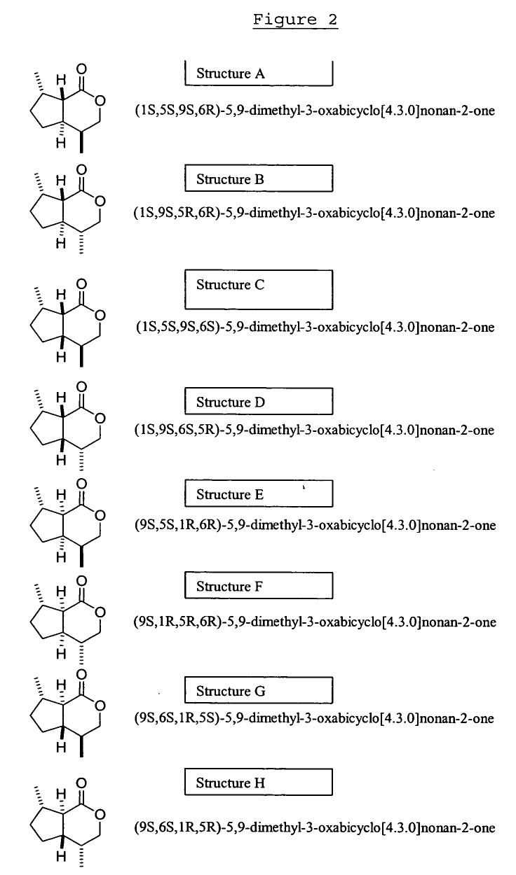

This invention is directed to a synthetic route for the stereospecific preparation of various isomers of DHN. Included within the products that can be obtained from the process of this invention is the diastereomeric form of DHN that is represented as Structure F in FIG. 2, (1R,5R,6R,9S)-5,9-dimethyl-3-oxabicyclo[4.3.0]nonan-2-one. This isomer may be obtained by applying the process of this invention to the naturally abundant cis, trans nepetalactone shown as Structure I(a) in FIG. 1.

Another product that may be obtained from the process of this invention is an approximately 1:1 diastereomeric mixture of the diastereomeric forms of DHN shown as Structures E and F in FIG. 2, or a mixture in which the DHN isomer of Structure F is present in an amount of at least 50%. This mixture of diastereomers may be prepared from naturally abundant trans, cis nepetalactone, shown as structure I(b) in FIG. 1. Other isomers of DHN may be obtained by the process of this invention, such as those available from cis, cis and trans, trans nepetalactone.

Nepetalactone may be viewed as a starting material in the process of this invention. It is a naturally occurring material that can be conveniently obtained in relatively pure form from the essential oils isolated by various means from plants of the genus Nepeta (catmints). Isolation of such oils is known in the art, and examples of methodology for oil extraction include without limitation steam distillation, organic solvent extraction, microwave-assisted organic solvent extraction, supercritical fluid extraction, mechanical extraction and enfleurage (initial cold extraction into fats followed by organic solvent extraction).

The essential oils isolated from different Nepeta species possess different proportions of each of the naturally-occurring stereoisomers of nepetalactone shown in FIG. 1 [Regnier, F. E. et al., Phytochemistry 6:1281-1289 (1967); DePooter, H. L. et al., Flavour and Fragrance Journal 3:155-159 (1988); Handjieva, N. V. and Popov, S. S., J. Essential Oil Res. 8:639-643 (1996)]. While the method of this invention may be performed upon the extracted oil containing a mixture of nepetalactones, it is preferred to first purify the separate fractions of nepetalactone in order to obtain products of high diastereomeric selectivity. It is found that cis, trans nepetalactone [FIG. 1(a)] is readily purified to a purity of about 95% or greater by fractional distillation of the extracted oil of nepeta.

Trans, cis nepetalactone [FIG. 1(b)] has been observed to undergo epimerization to the cis, trans stereoisomer upon heating, so distillation is not a preferred method for purifiying the trans, cis nepetalactone isomer. It has been found, however, that fractional crystallization is highly effective at preparing trans, cis nepetalactone at purities greater than 99%.

Cis, trans and trans, cis nepetalactones are by far the most prevalent specific stereoisomers occurring in nature that are derivable from the plant genus nepeta, and synthesis routes from naturally occurring sources are always more desirable. The cis, cis and trans, trans forms of nepetalactone may also be used in the process of this invention, however.

The nepetalactone used in the process of this invention may thus be provided by extraction or other means, and may be a mixture of isomers or purified. Regardless of its source or extent of purity, the nepeatalactone is contacted in the process of this invention with an aqueous base. Suitable bases include alkali metal, alkaline earth metal, and ammonium hydroxides. Sodium, potassium, lithium, calcium, magnesium, ammonium, and tetra-alkyl ammonium hydroxides are preferred. Sodium hydroxide is most preferred.

Preferably, the nepetalactone is first dissolved in a water-soluble aprotic solvent to form a solution. Representative solvents include tetrahydrofuran (THF), acetone, dimethylformamide, dimethylsulfoxide, dioxane, and dimethoxyethane, among others, and mixtures thereof. THF is preferred. The resulting solution is then dispersed with agitation in aqueous base. As the reaction proceeds while stirring, a homogeneous aqueous solution is formed.

It has been found advantageous to remove any reaction impurities at this stage because of the potential to interfere with crystallization of nepetalic acid in subsequent steps. Thus the basic solution formed as described above may then be subjected to extraction with one or more aliquots of an organic solvent such as ethyl acetate, hexane, dichloromethane, or diethylether, among others, and mixtures thereof. Preferred is ethyl acetate.

The step of forming a basic mixture is then followed by a step of acidification with an acid to form nepetalic acid. The extracted aqueous solution, as described above, is in this step subjected to gradual acidification to a pH below about 4, preferably to a pH of about 3 or below. Acidification is preferably achieved using a strong mineral acid, such as hydrochloric, nitric, or sulfuric acids, although it is preferred to use moderate concentrations thereof such as 1 molar rather than concentrated acid. The originally clear solution will turn opaque white after addition of the acid. The pH should be maintained above 1.

The thus acidified solution may then if desired be treated again with one or more aliquots of an organic solvent such as ethyl acetate, hexane, dichloromethane, or diethylether, among others, and mixtures thereof. Preferred is ethyl acetate. The organic extracts are then combined and contacted with an inorganic drying agent such as sodium sulfate to remove any residual moisture. The organic solvent is then removed by any convenient means; application of vacuum is satisfactory.

In the case of the single diastereomer cis, trans nepetalic acid (Structure IV, supra) prepared according to the embodiment hereof wherein cis, trans nepetalactone is the starting material, the resulting oil will crystallize upon standing at room temperature, and may be cooled to accelerate the process. It is found convenient to subject the oil to trituration with a hydrocarbon solvent, or mixture. Petroleum ether is found satisfactory.

In an alternative embodiment hereof, wherein trans, cis nepetalactone is subjected to the process of this invention, the resulting product oil is a diastereomeric mixture of carboxy aldehydes, shown here as Structures V(a) and V(b) 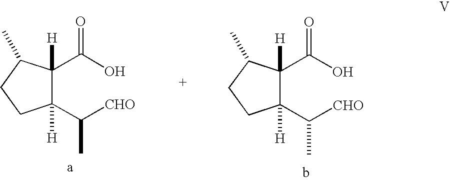
The diastereomeric mixture depicted in Structures V(a) and V(b) does not undergo crystallization.

In a further step in the process of the invention, nepetalic acid made as described above is subjected to deprotonation, and to reduction of the product thereof to DHN. For this purpose, the nepetalic acid may, in one embodiment, be contacted with a non-aqueous base such as a hydride to effect deprotonation at a temperature in the range of 0° C. to room temperature (e.g. about 25° C.); room temperature is found to be satisfactory. Suitable hydrides to be used for this purpose include alkali metal hydrides, particularly Na, K, or Li hydride. LiAlH₄ should be expressly avoided. Preferred is KH. Also useful for the deprotonation are amines, particularly triethylamine.

Preferably the deprotonation step, and more preferably also the subsequent reduction, takes place in a nepetalic acid solution. Suitable solvents are aprotic solvents which solvate nepetalic acid and are unreactive towards the base employed. Suitable solvents include THF and dimethoxy ethane. THF is preferred.

Following the deprotonation step, the resulting salt is contacted with a reducing agent to form the DHN product. Suitable reducing agents include borohydrides and dialkylboranes such as lithium borohydride, potassium borohydride, zinc borohydride, diisobutylaluminum hydride, bis(methoxyethoxy)aluminohydride, tetrabutylammonium hydride, lithium tri(t-butoxy)aluminohydride, sodium cyanoborohyride, tetrabutylammonium cyanoborohyride, zinc cyanoborohyride, lithium triethylborohydride, lithium tributylborohydride, potassium tributylborohydride, tetrabutylammonium tributylborohydride, cuprous bisdiphenylphosphineborohydride, cuprous bisdiphenylphosphinecyanoborohyride, potassium triisopropoxyborohydide, and tetrabutylammonium triacetoxyborane.

In general, tetraalkylammonium cations can be used in the reducing agent in place of the alkalai metal cations like sodium or potassium, and may in some instances give better performance than the metal counterparts because of the lipophilic nature. Tetrabutylammonium is a common and commercially-available cation, but a smaller tetraalkylammonium group is suitable as well. Similarly, a tributylborohydride may be used as a trialkylborohydride, but other trialkylborohydrides such as methyl, ethyl and n-propyl are suitable as well. Lithium aluminumhydride, aluminum hydride, aluminum chlorohydrides, borane, and borane complexes (such as borane-THF, borane-dimethylsulfide complex, or borane-amine complexes) have been found to give less than desired performance as the reducing agent. The preferred reducing agent is an alkali metal borohydride such as NaBH₄.

In a preferred embodiment, the separate deprotonation step is eliminated by employing an excess of the reducing agent (such as NaBH₄)—that is, more than one equivalent, preferably slightly more than two equivalents of the reducing agent to effect both the deprotonation and reduction in a single step.

It is found in the practice of the invention that methanol is an excellent solvent for the reactants but is highly reactive at room temperature with the NaBH₄. This turns out to be beneficial. When methanol is employed as the solvent, the solution of nepetalic acid must be cooled to less than room temperature (e.g. 25° C.), such as to about 0° C., prior to the addition of the NaBH₄. After the reaction is complete, and the solution is allowed to warm, the methanol solvent will react with the remaining NaBH₄, thus effectively cleaning the reaction mixture, and eliminating the need to employ exact stoichiometric amounts of the NaBH₄.

Upon completion of the reaction, the dihydronepetalactone diastereomeric product may be purified by distillation or by crystallization, or by preparative liquid chromatography.

Except where otherwise indicated, the chemical reactions of the process of this invention may conveniently be performed at room temperature, without special measures taken to heat or to cool. Thus temperatures in the range of 20-30° C. have been found to be satisfactory. In general, heating above 30° C. should be avoided in order to avoid undesirable side reactions. Temperatures below 20° C., down to 0° C., may, however, be employed for the purposes described above or if otherwise desired. There is no limitation on the specific methods and means by which the process of the present invention may be carried out. Batch processing as well as continuous processing using commonly employed equipment are both viable processing routes.

The process of this invention is a high yield reaction, with typical yields being in the range of 85-90% of the desired product. In the case in which cis, trans nepetalactone is subjected to the process of this invention to form first Structure IV and then Structure F (in FIG. 2), the yield applies to the single diastereomer. In the case in which trans, cis nepetalactone is subjected to the process of this invention, the product is an approximately 1:1 diastereomeric mixture of Structures E and F (in FIG. 2). This diastereomeric mixture is not separable by ordinary means.

The DHN produced by the process of this invention may be used for a multiplicity of purposes, such as use in an effective amount for the repellency of various insect species, or as a fragrance compound in a perfume composition, or as a topical treatment for skin. For example, the compounds hereof may be applied in a topical manner to human or animal skin, fur or feathers, or to growing plants or crops, to impart insect repellency or a pleasant odor or aroma.

DHN is typically used for such purposes in a composition in which the DHN is admixed with a carrier. Suitable carriers include any one of a variety of commercially available organic and inorganic liquid, solid, or semi-solid carriers or carrier formulations usable in formulating skin or insect repellent products. When formulating a skin product or topical insect repellent, it is preferred to select a dermatologically acceptable carrier. For example the carrier may include water, alcohol, silicone, petrolatum, lanolin or many of several other well known carrier components. Examples of organic liquid carriers include liquid aliphatic hydrocarbons (e.g., pentane, hexane, heptane, nonane, decane and their analogs) and liquid aromatic hydrocarbons.

Examples of other liquid hydrocarbons include oils produced by the distillation of coal and the distillation of various types and grades of petrochemical stocks, including kerosene oils that are obtained by fractional distillation of petroleum. Other petroleum oils include those generally referred to as agricultural spray oils (e.g., the so-called light and medium spray oils, consisting of middle fractions in the distillation of petroleum and which are only slightly volatile). Such oils are usually highly refined and may contain only minute amounts of unsaturated compounds. Such oils, moreover, are generally paraffin oils and accordingly can be emulsified with water and an emulsifier, diluted to lower concentrations, and used as sprays. Tall oils, obtained from sulfate digestion of wood pulp, like the paraffin oils, can similarly be used. Other organic liquid carriers can include liquid terpene hydrocarbons and terpene alcohols such as alpha-pinene, dipentene, terpineol, and the like.

Other carriers include silicone, petrolatum, lanolin, liquid hydrocarbons, agricultural spray oils, paraffin oil, tall oils, liquid terpene hydrocarbons and terpene alcohols, aliphatic and aromatic alcohols, esters, aldehydes, ketones, mineral oil, higher alcohols, finely divided organic and inorganic solid materials. In addition to the above-mentioned liquid hydrocarbons, the carrier can contain conventional emulsifying agents which can be used for causing the dihydronepetalactone compounds to be dispersed in, and diluted with, water for end-use application. Still other liquid carriers can include organic solvents such as aliphatic and aromatic alcohols, esters, aldehydes, and ketones. Aliphatic monohydric alcohols include methyl, ethyl, normal-propyl, isopropyl, normal-butyl, sec-butyl, and tert-butyl alcohols. Suitable alcohols include glycols (such as ethylene and propylene glycol) and pinacols. Suitable polyhydroxy alcohols include glycerol, arabitol, erythritol, sorbitol, and the like. Finally, suitable cyclic alcohols include cyclopentyl and cyclohexyl alcohols.

Conventional aromatic and aliphatic esters, aldehydes and ketones can be used as carriers, and occasionally are used in combination with the above-mentioned alcohols. Still other liquid carriers include relatively high-boiling petroleum products such as mineral oil and higher alcohols (such as cetyl alcohol). Additionally, conventional or so-called “stabilizers” (e.g., tert-butyl sulfinyl dimethyl dithiocarbonate) can be used in conjunction with, or as a component of, the carrier or carriers comprising the compositions of the present invention.

Desirable properties of a topical insect repellent article include low toxicity, resistance to loss by water immersion or sweating, low or no odor or at least a pleasant odor, ease of application, and rapid formation of a dry tack-free surface film on the host's skin. In order to obtain these properties, the formulation for a topical insect repellent article should permit insect-infested animals (e.g., dogs with fleas, poultry with lice, cows with horn flies or ticks, and humans) to be treated with an insect repellent (including a composition thereof) by contacting the skin, fur or feathers of such an animal with an effective amount of the repellent for repelling the insect from the animal host.

Dispersing the repellent into the air or dispersing the repellent as a liquid mist or incorporated into a powder or dust will thus permit the repellent to fall on the desired host surfaces. It may also be desirable to formulate an insect repellent by combining a DHN to form a composition with a fugitive vehicle for application in the form of a spray. Such a composition may be an aerosol composition adapted to disperse the dihydronepetalactone into the atmosphere by means of a compressed gas, or a mechanical pump spray. Likewise, directly spreading of a liquid/semi-solid/solid repellent on the host is an effective method of contacting the surface of the host with an effective amount of the repellent.

DHN may also be combined with other insect repellent substances such as N,N-diethyl-meta-toluamide (DEET).

In addition to a DHN, an insect repellent composition may also include one or more essential oils and/or active ingredients of essential oils. “Essential oils” are defined as any class of volatile oils obtained from plants possessing the odor and other characteristic properties of the plant. Examples of useful essential oils include: almond bitter oil, anise oil, basil oil, bay oil, caraway oil, cardamom oil, cedar oil, celery oil, chamomile oil, cinnamon oil, citronella oil, clove oil, coriander oil, cumin oil, dill oil, eucalyptus oil, fennel oil, ginger oil, grapefruit oil, lemon oil, lime oil, mint oil, parsley oil, peppermint oil, pepper oil, rose oil, spearmint oil (menthol), sweet orange oil, thyme oil, turmeric oil, and oil of wintergreen. Examples of active ingredients in essential oils are: citronellal, methyl salicylate, ethyl salicylate, propyl salicylate, citronellol, safrole, and limonene.

The insects that may be repelled by the compounds of this invention may include any member of a large group of invertebrate animals characterized, in the adult state (non-adult insect states include larva and pupa) by division of the body into head, thorax, and abdomen, three pairs of legs, and, often (but not always) two pairs of membranous wings. This definition therefore includes a variety of biting insects (e.g. ants, bees, chiggers, fleas, mosquitoes, ticks, wasps), biting flies [e.g. black flies, green head flies, stable flies, horn flies (haematobia irritans)], wood-boring insects (e.g. termites), noxious insects (e.g. houseflies, cockroaches, lice, roaches, wood lice), and household pests (e.g. flour and bean beetles, dust mites, moths, silverfish, weevils). A host from which it may be desired to repel an insect may include any plant or animal (including humans) affected by insects. Typically, hosts are considered to be insect-acceptable food sources or insect-acceptable habitats.

In another embodiment, a DHN may be used as a fragrance compound or in a fragrance composition, and be applied in a topical manner to human or animal skin or hair to impart a pleasing fragrance, as in skin lotions and perfumes.

Particularly because of the pleasant aroma associated with the compounds hereof, a further embodiment of this invention is one in which one or more DHNs are formulated into a composition for use as a product that is directed to other fundamental purposes. The fragrance and/or insect repellency of these products will be enhanced by the presence therein of compound(s) of this invention. Included among such products (but not thereto limited) are colognes, lotions, sprays, creams, gels, ointments, bath and shower gels, foam products (e.g., shaving foams), makeup, deodorants, shampoo, hair lacquers/hair rinses, and personal soap compositions (e.g., hand soaps and bath/shower soaps). The compound(s) may of course be incorporated into such products simply to impart a pleasing aroma. Any means of incorporation such as is practiced in the art is satisfactory.

A corresponding aspect of the wide variety of products discussed above is a further alternative embodiment of this invention, which is a process for fabricating a composition of matter, a topical treatment for skin, or an article of manufacture, by providing as the composition, or incorporating into the composition, skin treatment or article, one or more DHNs, or a mixture of stereoisomers thereof. Such products, and the method and process described above, illustrate the use of a DHN as a fragrance compound or perfume, or in a fragrance composition or formulation, or in a topical treatment for skin, or in an article of manufacture.

A composition containing compound(s) of this invention prepared as an insect repellent, fragrance product, or other personal care product may also contain other therapeutically or cosmetically active adjuvants or ingredients as are typical in the personal care industry. Examples of these include fungicides, sunscreening agents, sunblocking agents, vitamins, tanning agents, plant extracts, anti-inflammatory agents, anti-oxidants, radical scavenging agents, retinoids, alpha-hydroxy acids, antiseptics, antibiotics, antibacterial agents, antihistamines; adjuvants such as thickeners, buffering agents, chelating agents, preservatives, gelling agents, stabilizers, surfactants, emolients, coloring agents, aloe vera, waxes, and penetration enhancers; and mixtures of any two or more thereof.

The amount of a compound of this invention contained in a composition will generally not exceed about 80% by weight based on the weight of the final product, however, greater amounts may be utilized in certain applications and this amount is not limiting. More preferably, a suitable amount of a compound will be at least about 0.001% by weight and preferably about 0.01% up to about 50% by weight; and more preferably, from about 0.01% to about 20% weight percent, based on the weight of the composition or article. Specific compositions will depend on the intended use.

In a further embodiment of this invention, a DHN is incorporated into an article to produce an insect repellent effect. Articles contemplated to fall within this embodiment include manufactured goods, including textile goods such as clothing, outdoor or military equipment as mosquito netting, natural products such as lumber, or the leaves of insect vulnerable plants.

In another embodiment of this invention, a DHN is incorporated into an article to produce a fragrance pleasing to some humans, or a DHN is applied to the surface of an object to impart an odor thereto. The particular manner of application will depend upon the surface in question and the concentration required to impart the necessary intensity of odor. Articles contemplated to fall within these embodiments include manufactured goods, including textile goods, air fresheners, candles, various scented articles, fibers, sheets, paper, paint, ink, clay, wood, furniture (e.g., for patios and decks), carpets, sanitary goods, plastics, polymers, and the like.

Other uses for or compositions of a DHN are as disclosed in U.S. 2003/062,357; U.S. 2003/079,786; and U.S. 2003/191,047, each of which is incorporated in its entirety as a part hereof.

The present invention is further described according to the following specific embodiments, but the scope hereof is not limited thereto.

All reactions and manipulations are carried out in a standard laboratory fume hood in standard laboratory glassware. Nepetalactones are obtained by steam distillation of commercially-available catnip oil from catmint, obtained from Berjé, (Bloomfield, N.J.). Cis,trans-nepetalactone is further purified by vacuum distillation and trans, cis-nepetalactone is purified by crystallization at 0° C. from petroleum ether and hexanes. All inorganic salts and organic solvents were obtained from VWR Scientific. All other reagents used in the examples were obtained from Sigma-Aldrich Chemical (Milwaukee, Wis.) and used as received. Determination of pH is done with pHydrion paper from Micro Essential Laboratory. The dihydronepetalactone products are purified by column chromatography and characterized by NMR spectroscopy. NMR spectra are obtained on a Bruker DRX Advance (500 MHz ¹H, 125 MHz ¹³C) using deuterated solvents obtained from Cambridge Isotope Laboratories.

### EXAMPLE 1

Nepetalic acid is prepared from cis, trans nepetalactone, (3S,4R,4aR,7S,7aR)-3-hydroxy-4,7-dimethylhexahydrocyclopenta[c]pyran-1(3H)-one, according to the following procedure.

A solution of cis-trans nepetalactone in 5 mL of tetrahydrofuran is treated with sodium hydroxide (1.0 g in 5 mL of water) resulting in initially a two-phase mixture, which becomes a homogeneous yellow solution after 1 hour. The basic solution so formed is extracted twice with fresh 20 mL aliquots of ethyl acetate. The aqueous layer from this extraction is acidified drop-wise with 1N HCl to pH=3, becoming a white heterogeneous mixture. The thus formed aqueous mixture is extracted twice with ethyl acetate and dried over anhydrous sodium sulfate. Removal of the solvent under vacuum results in a yellow oil, which is triturated with petroleum ether (100 mL) and allowed to crystallize to a white solid on standing. The white solid is filtered, washed with cold petroleum ether (20 mL) and dried under high vacuum to afford nepetalic acid (1.9 g, 69%) with a melting point of 67° C. [lit.: 71° C., J. Org. Chem., Vol. 46, No. 16 (1981), 3302-3305]. The absolute stereochemistry of the product is verified by single crystal analysis and is consistent with the single diastereomer, (3S,4R,4aR,7S,7aR)-3-hydroxy-4,7-dimethylhexahydrocyclopenta[c]pyran-1(3H)-one) (Structure IV, supra).

An oven-dried 500 mL liter three-necked round-bottom flask is cooled to room temperature under a steady stream of nitrogen. A solution of 5 g of the so prepared nepetalic acid in 100 mL of methanol is added to the flask and then cooled to 0° C. To that solution, 1.45 g of sodium borohydride is added portion-wise over a period of 30 minutes while under a steady stream of nitrogen to 0° C. After the addition is complete, the solution is warmed to room temperature. After 3 hours, the reaction is acidified by drop-wise addition of 1N HCl to pH=3.0, and the resulting solution is transferred to separatory funnel and extracted with dichloromethane (30 mL) three times. The combined organics are dried over anhydrous sodium sulfate. Removal of the solvent under vacuum affords the product as a pale oil (4.35 g), which is purified by column chromatography on silica gel eluting with 5% ethyl acetate in hexanes. The product-containing fractions are identified by TLC analysis, combined and the solvent is removed under vacuum to afford the product (2.64 g). ¹H and ¹³C NMR analysis of the product confirm the structure of (1R,5R,6R,9S)-5,9-dimethyl-3-oxabicyclo[4,3,0]nonan-2-one (Structure F in FIG. 2).

### EXAMPLE 2

Nepetalic acid is prepared from trans, cis nepetalactone by the identical procedure employed for nepetalic acid used in Example 1 with the exception that trans, cis nepetalactone is used in place of cis-trans nepetalactone. The following amounts of reagents and solvents are used:
8.93 g of trans, cis nepetalactone
3.2 g of sodium hydroxide
20 mL of THF
20 mL of water

9 g of product is obtained as a pale yellow oil and is used without further purification. NMR analysis of the product obtained is consistent with a 1:1 mixture of (1S,2S,5R)-2-methyl-5-[(1R)-1-methyl-2-oxoethyl]cyclopentanecarboxylic acid and (1S,2S,5R)-2-methyl-S-[(1S)-1-methyl-2-oxoethyl]cyclopentanecarboxylic acid, as represented by the diastereomers of Structures V(a) and V(b).

An oven-dried 50 mL liter three-necked round-bottomed flask is cooled to room temperature under a steady stream of nitrogen. A solution of 184 mg of the nepetalic acid diastereomeric mixture so prepared in 10 mL of methanol is added to the flask and then cooled to 0° C. To that solution, 54 mg of sodium borohydride is added in one portion while under a steady stream of nitrogen to 0° C., and the contents are then warmed to room temperature. After 3 hours, the reaction is acidified by drop-wise addition of 1N HCl to pH=3.0, and the resulting solution is transferred to separatory funnel and extracted with dichloromethane (10 mL) three times. The combined organics are dried over anhydrous sodium sulfate. Removal of the solvent under vacuum affords the product as a clear oil (171 mg), which is purified by column chromatography on silica gel eluting with 5% ethyl acetate in hexanes. The product-containing fractions are identified by TLC analysis, combined and the solvent is removed under vacuum to afford the product (64 mg). ¹H and ¹³C NMR analysis of the product confirm the structure of (1R,5R,6R,9S)-5,9-dimethyl-3-oxabicyclo[4,3,0]nonan-2-one (Structure F).

### EXAMPLE 3

The product of Example 1 is evaluated for insect repellency in a comparison test with DHN stereoisomers prepared according to prior-art methods, and against the major commercial insect repellent composition, DEET (N,N-diethyl-m-toluamide). As a control, neat iso-propanol (IPA) is employed as well.

The DHN contained in the composition tested as Example 1 is the single diastereomer of Structure F.

The DHN contained in the composition tested as Comparative Example 1 is prepared according to the methods of Hallahan, op.cit., and Manzer, op. cit, using purified cis, trans nepetalactone, purified as described hereinabove. The resulting product is a 7:1 mixture of the diastereomers shown as Structures E and F, respectively, in FIG. 2.

The DHN contained in the composition tested as Comparative Example 2 is prepared according to the methods of Hallahan, op.cit., and Manzer, op. cit, using purified trans, cis nepetalactone, purified as described hereinabove. The resulting product is a single diastereomer shown as Structure B of FIG. 2.

The composition tested as Comparative Example 1 thus contains a mixture of diastereomers, one of which is the diastereomer of Structure F present as a minor component. The composition tested as Example 1, by contrast, contains the diastereomer of Structure F as the only active component.

Repellency is determined against Aedes aegypti mosqutioes in the in vitro Gupta box landing assay. In this method a chamber contains 5 wells, each covered by a Baudruche (animal intestine) membrane. Each well is filled with bovine blood, containing sodium citrate (to prevent clotting) and ATP (72 mg ATP disodium salt per 26 ml of blood), and heated to 37° C. A volume of 25 μl of isopropyl alcohol (IPA) containing one test specimen or control is applied to each membrane. The concentrations are all 1% in IPA except where otherwise indicated. Controls are either neat IPA, an untreated membrane surface, or a membrane surface treated with a 1% solution of DEET.

After 5 min, approximately 250 4-day-old female Aedes aegypti mosquitoes are introduced into the chamber. The number of mosquitoes probing the membranes for each treatment is recorded at 2 minute intervals over 20 minutes. Each datum represents the mean of three replicate experiments.

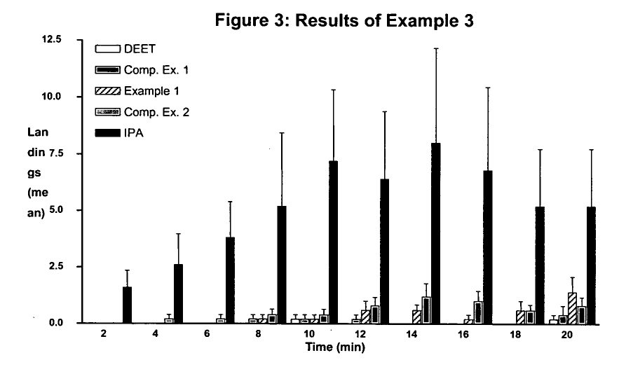

Results are shown in FIG. 3 in which mean number of landings is recorded on the vertical scale, and elapsed time is recorded on the horizontal scale. It may be seen from FIG. 3 that DHN Structure F (as contained in the composition tested as Example 1) compared well in repellent efficacy with the DHN materials prepared by the various prior-art methods.

Where a composition or method of this invention is stated or described as comprising, including, containing, having, being composed of or being constituted by certain components or steps, it is to be understood, unless the statement or description explicitly provides to the contrary, that one or more components or steps other than those explicitly stated or described may be present in the composition or method. In an alternative embodiment, however, the composition or method of this invention may be stated or described as consisting essentially of certain components or steps, in which embodiment components or steps that would materially alter the principle of operation or the distinguishing characteristics of the composition or method would not be present therein. In a further alternative embodiment, the composition or method of this invention may be stated or described as consisting of certain components or steps, in which embodiment components or steps other than those as stated would not be present therein.

Where the indefinite article “a” or “an” is used with respect to a statement or description of the presence of a component in a composition, or a step in a method, of this invention, it is to be understood, unless the statement or description explicitly provides to the contrary, that the use of such indefinite article does not limit the presence of the component in the composition, or of the step in the method, to one in number.

1. A process for preparing a dihydronepetalactone, represented schematically as Structure II, comprising
(a) contacting nepetalactone, represented schematically as Structure I, with an aqueous base;
(b) contacting the product of step (a) with an acid to form nepetalic acid, represented schematically as Structure III; and
(c) contacting the nepetalic acid with a reducing agent to form dihydronepetalactone.

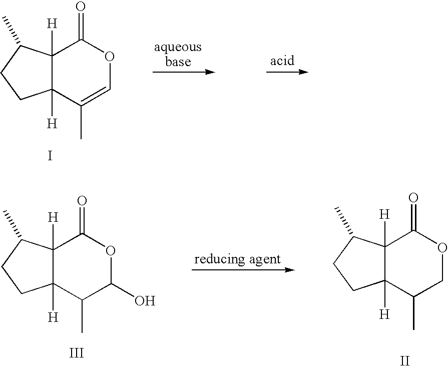

2. The process of claim 1 wherein the acid is a mineral acid.

3. The process of claim 1 wherein the reducing agent is an alkali metal borohydride.

4. The process of claim 1 further comprising a step of contacting the nepetalic acid with a non-aqueous base prior to contact with the reducing agent.

5. The process of claim 1, which is run in methanol at a temperature of less than 25° C.

6. The process of claim 1 wherein the nepetalactone is cis, trans nepetalactone ((3S,4R,4aR,7S,7aR)-3-hydroxy-4,7-dimethylhexahydrocyclopenta[c]pyran-1(3H)-one), represented by Structure I(a), 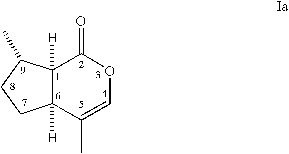
and a dihydronepetalactone so produced is (9S,1R,5R,6R)-5,9-dimethyl-3-oxabicyclo[4.3.0]nonan-2-one, represented by Structure F

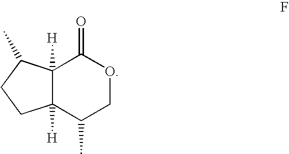

7. A composition of matter comprising (a-1) the single diastereomer of dihydronepetalactone (9S,1R,5R,6R)-5,9-dimethyl-3-oxabicyclo[4.3.0]nonan-2-one, or (a-2) a mixture of diastereomers of dihydronepetalactone whereof at least 50% thereof is (9S,1R,5R,6R)-5,9-dimethyl-3-oxabicyclo[4.3.0]nonan-2-one; and (b) a carrier.

8. The composition of claim 7 which comprises dihydronepetalactone in an amount of from about 0.001% to about 80% by weight of the total weight of the composition.

9. The composition of claim 7 wherein the carrier is selected from the group consisting of silicone, petrolatum, lanolin, liquid hydrocarbons, agricultural spray oils, paraffin oil, tall oils, liquid terpene hydrocarbons and terpene alcohols, aliphatic and aromatic alcohols, esters, aldehydes, ketones, mineral oil, higher alcohols, finely divided organic and inorganic solid materials.

10. The composition of claim 7 wherein the carrier comprises an aerosol composition adapted to disperse the dihydronepetalactone into the atmosphere by means of a compressed gas.

11. The composition of claim 7 wherein the carrier comprises a powder or dust.

12. The composition of claim 7, which further comprises an adjuvant, an insect repellent compound that is not a dihydronepetalactone, or a mixture thereof.

13. The composition of claim 12 wherein the adjuvant is selected from the group consisting of thickeners, buffering agents, chelating agents, preservatives, fragrances, antioxidants, gelling agents, stabilizers, surfactants, emolients, coloring agents, aloe vera, waxes, and therapeutically or cosmetically active ingredients.

14. The composition of claim 7 which is in the form of a cologne, a lotion, a spray, a cream, a gel, an ointment, a bath or shower gel, a foam product, makeup, a deodorant, shampoo, a hair lacquer or rinse or a personal soap.

15. The composition of claim 7 which is incorporated into an article which is selected from the group consisting of air freshener, a candle, a scented article, a fiber, a sheet, cloth, paper, paint, ink, clay, wood, furniture, netting, carpeting, sanitary goods, a plastic, and a polymer.

16. A method of repelling one or more insects from a human, animal or inanimate host comprising exposing the insects to a composition comprising (a-1) the single diastereomer of dihydronepetalactone (9S,1R,5R,6R)-5,9-dimethyl-3-oxabicyclo[4.3.0]nonan-2-one, or (a-2) a mixture of diastereomers of dihydronepetalactone whereof at least 50% thereof is (9S,1R,5R,6R)-5,9-dimethyl-3-oxabicyclo[4.3.0]nonan-2-one; and (b) a carrier.

17. The method of claim 16 wherein the composition is in the form of a cologne, a lotion, a spray, a cream, a gel, an ointment, a bath or shower gel, a foam product, makeup, a deodorant, shampoo, a hair lacquer or rinse or a personal soap.

18. The method of claim 16 wherein the carrier comprises an aerosol composition adapted to disperse the dihydronepetalactone into the atmosphere by means of a compressed gas.

19. The method of claim 16 wherein the carrier comprises a powder or dust.

20. The method of claim 16 wherein the insect(s) are selected from one or more members of the group consisting of black flies, green head flies, stable flies, horn flies, mosquitoes, ticks, chiggers, fleas and lice.

21. A method of fabricating an insect repellent composition, or an insect repellent article of manufacture, comprising forming the composition from, or incorporating into the article, the single diastereomer of dihydronepetalactone (9S,1R,5R,6R)-5,9-dimethyl-3-oxabicyclo[4.3.0]nonan-2-one, or a mixture of diastereomers of dihydronepetalactone whereof at least 50% thereof is (9S,1R,5R,6R)-5,9-dimethyl-3-oxabicyclo[4.3.0]nonan-2-one.
